(19) United States
(12) Patent Application Publication
(10) Pub. No.: US 2024/0382445 A1
(43) Pub. Date: Nov. 21, 2024

(54) METHODS OF ADJUVANT THERAPY

(71) Applicant: Lipid3 Technologies Ltd., HALIFAX (CA)

(72) Inventor: Jenna Ritter, Halifax (CA)

(73) Assignee: Lipid3 Technologies Ltd., HALIFAX, NS (CA)

(21) Appl. No.: 18/408,485

(22) Filed: Jan. 9, 2024

Related U.S. Application Data
(63) Continuation of application No. 18/518,319, filed on Nov. 22, 2023, now abandoned.
(60) Provisional application No. 63/502,619, filed on May 16, 2023.

(51) Int. Cl. A61K 31/202; A61K 45/06

(52) U.S. Cl. CPC A61K 31/202; A61K 45/06

(57) ABSTRACT

The present disclosure relates to methods of treating diseases. More particularly, the disclosure relates to methods of adjuvant therapy. Disclosed herein are methods of promoting 8-hydroxyoctanoic acid (8-HOA) production and thereby treating diseases. The method includes administering for a first period of time a first dosage form including eicosapentaenoic acid (EPA) to a patient. After expiration of the first period of time, the patient is then administered for a second period of time a second dosage form including gamma-linolenic acid (GLA).

### CROSS-REFERENCE TO RELATED APPLICATIONS

This application is a continuation of U.S. patent application Ser. No. 18/518,319, filed Nov. 22, 2023, and entitled METHODS OF ADJUVANT THERAPY, which in turn claims the benefit of U.S. Provisional Patent Application Ser. No. 63/502,619 filed May 16, 2023, and entitled METHODS OF ADJUVANT THERAPY, the entire contents of both of which are incorporated by reference herein.

### TECHNICAL FIELD

The present disclosure relates to methods of treating diseases. More particularly, the disclosure relates to methods of adjuvant therapy.

### BACKGROUND

Significant advances have been made in the treatment of cancers, inflammatory diseases, and other diseases. However, there continues to be a need for development of new treatments and/or increasing the effectiveness of existing treatments for cancers, inflammatory diseases, and other diseases.

### BRIEF DESCRIPTION OF THE DRAWINGS

The embodiments disclosed herein will become more fully apparent from the following description and appended claims, taken in conjunction with the accompanying drawings.

FIG. 1 illustrates graphically the formation and fate of GLA in humans.

### DETAILED DESCRIPTION

The present disclosure relates to methods of treating diseases. More particularly, the disclosure relates to methods of adjuvant therapy. It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

Applicant has discovered that by timing the dosing of certain fatty acids, the fatty acids can increase the effectiveness of the primary treatment for certain diseases. Applicant has discovered a method of promoting 8-hydroxyoctanoic acid (8-HOA) production in vivo in patients. The method includes administering for a first period of time a first dosage form including eicosapentaenoic acid (EPA) to a patient. After expiration of the first period of time, the patient is then administered for a second period of time a second dosage form including gamma-linolenic acid (GLA). Accumulating a baseline level of EPA in the cells of the patient, prior to administering GLA, promotes production of 8-HOA instead of arachidonic acid (AA) and its downstream products.

8-HOA is an 8-carbon fatty acid with a hydroxyl group at its terminal end and is formed in cells through COX-2 peroxidation of dihomo-linolenic acid (DGLA, 20:3n-6). Structure of 8-hydroxyoctanoic acid (8-HOA):

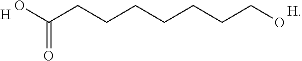

Gamma linoleic acid (GLA, 18:3n-6) is an omega-6 fatty acid that is found in some plant oils, primarily evening primrose and borage oils. It is also formed via metabolism of linoleic acid (LA, 18:2n-6) by Δ6-desaturase. Unlike LA, dietary GLA can bypass the rate-limiting desaturation step by Δ6-desasturase and is rapidly converted to DGLA. DGLA can further be desaturated to form arachidonic acid (AA, 20:4n-6) but the enzyme responsible for this, Δ5-desaturase (D5D), has limited activity which prevents full conversion of DGLA to AA (Wang X, Lin H, Gu Y (2012) Multiple roles of dihomo-linolenic acid against proliferation diseases. Lipids in Health and Disease, 11:25, the contents of which are incorporated herein by reference, referred to hereinafter as “Wang et al. 2012”). DGLA is found in trace amounts in breast milk and some meats but is not typically consumed as part of an adult diet.

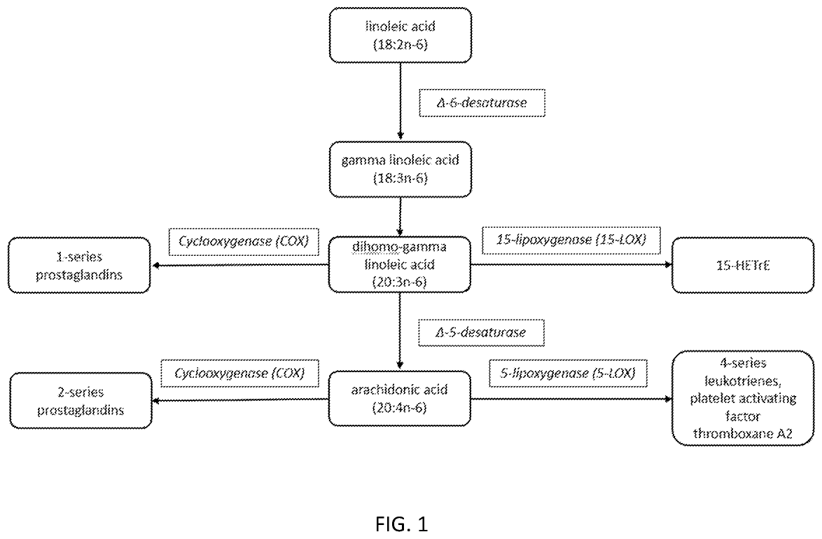

FIG. 1 illustrates graphically the formation and fate of GLA in humans. DGLA and AA are metabolized by the cyclooxygenase (COX) enzymes, lipid peroxidation enzymes, to form 1-series prostaglandins (PG1) and 2-series prostaglandins-2 (PG2). PG1 are generally considered to have anti-inflammatory properties and PG2 are generally thought to be pro-inflammatory (Wang et al. 2012). COX-2 catalyzed peroxidation of DGLA also leads to the formation of 8-HOA.

To increase levels of 8-HOA it is necessary to prevent conversion of DGLA to AA, which can be accomplished by inhibiting the activity of D5D. Eicosapentaenoic acid (EPA, 20:5n-3) may be used to inhibit D5D. By first increasing the baseline presence of EPA in a patient and then secondly administering GLA, GLA conversion to AA can be inhibited and in vivo 8-HOA production increased.

Increased in vivo 8-HOA production can be used to treat or aid the treatment of several diseases. For example, 8-HOA may promote the effectiveness of COX-2 inhibitors and histone deacetylase (HDAC) inhibitors.

In certain embodiments, the methods disclosed herein include a method of adjunctive therapy, wherein the method includes administering for a first period of time a first dosage form comprising eicosapentaenoic acid (EPA) to a patient diagnosed with a cyclooxygenase-2 (Cox-2) enzyme over-expressing disease, wherein said patient is receiving or will receive a primary treatment for the Cox-2 enzyme over-expressing disease. The method also includes, after expiration of the first period of time, administering for a second period of time a second dosage form comprising gamma-linolenic acid (GLA) to the patient.

The Cox-2 enzyme over-expressing disease may involve an inflammatory disorder or a cancer. The method may include determining or having determined that the patient has a disease that over-expresses the Cox-2 enzyme. Likewise, the primary treatment may involve the patient receiving an non-steroidal anti-inflammatory drug (NSAID).

In certain embodiments, the methods disclosed herein include a method of adjunctive therapy, wherein the method includes administering for a first period of time a first dosage form comprising eicosapentaenoic acid (EPA) to a patient, wherein said patient is receiving or will receive an histone deacetylase (HDAC) inhibitor. The method includes after expiration of the first period of time, administering for a second period of time a second dosage form comprising gamma-linolenic acid (GLA) to the patient.

The patient receiving or to receive the HDAC inhibitor may have been diagnosed with a cancer or chronic obstructive pulmonary disease (COPD). The method may include determining or having determined that the patient has a disease responsive to an HDAC inhibitor.

In the methods disclosed herein, the EPA may be provided as an ester, triglyceride (including re-esterified triglyceride), free fatty acid, phospholipid or other polar lipid, or combinations thereof. The EPA may also be provided as a precursor or prodrug of EPA that metabolizes in vivo to form EPA in the patient.

In the methods disclosed herein, the GLA may be provided as an ester, triglyceride (including re-esterified triglyceride), free fatty acid, phospholipid or other polar lipid, or combinations thereof. The GLA may also be provided as a precursor or prodrug of GLA that metabolizes in vivo to form GLA in the patient.

In the methods disclosed herein, the first period of time may be at least two weeks, at least six week, or six weeks to three months.

In the methods disclosed herein, the first period of time may extend until the patient's red blood cells have an average percent EPA at or above a specified level.

In the methods disclosed herein, the daily dose of EPA during the first period of time may be 100 mg to 500 mg, 500 mg to 2000 mg, or 50 mg to 2000 mg.

In the methods disclosed herein, the daily dose of GLA during the second period of time may be 100 mg to 500 mg, 500 mg to 2000 mg, 2000 mg to 4000 mg, or 50 mg to 4000 mg.

In the methods disclosed herein, the daily dose of EPA during the second period of time may be 100 mg to 500 mg, 500 mg to 2000 mg, or 50 mg to 2000 mg.

In the methods disclosed herein, the first dosage form may substantially exclude linolenic acid (LA), gamma-linolenic acid (GLA), and dihomo-gamma-linolenic acid (DGLA).

In the methods disclosed herein, the second dosage form may not be administered to the patient during the first period of time.

In the methods disclosed herein, the first dosage form, the second dosage form, or both, further include docosahexaenoic acid (DHA).

In the methods disclosed herein, the second dosage form may include EPA.

In the methods disclosed herein, the first dosage form may continue to be administered during the second period of time. Or stated another way, the first dosage form may be administered with the second dosage form during the second period of time. When co-administered during the second period of time, the first dosage form may be administered 0-8 hours before administering the second dosage form.

In the methods disclosed herein, the first period of time may precede initiation of the primary treatment. For example, upon diagnosis of the disease, the patient may be administered the first dosage form to increase in vivo levels of EPA prior to administering the second dosage form and beginning the primary treatment.

In the methods disclosed herein, the second period of time is for at least the duration of the primary treatment.

In the methods disclosed herein, the patient is a mammal, a human, a canine, or a feline.

As used herein, “administering” encompasses either (i) administering a compound, or a pharmaceutical compositions comprising the compound, directly to isolated cells or to an animal, or (ii) administering to cells or an animal another agent to cause the presence or formation of the compound inside the cells or the animal. Accordingly, the “another agent” may be administered in a sufficient amount to achieve a therapeutically effective amount of the compound inside the cells or the animal.

In any of the embodiments disclosed herein, the administering may include orally administering. Other enteral routes, such as buccal, sublingual, and rectal are also possible. Parenteral routes of administration are also possible, such as topical, intracerebroventricular, intravenous, intramuscular, subcutaneous, and transdermal.

It will be apparent to those having skill in the art that many changes may be made to the details of the above-described embodiments without departing from the underlying principles of the invention.

1. A method of adjunctive therapy, the method comprising:
administering for a first period of time a first dosage form comprising eicosapentaenoic acid (EPA) and substantially excluding linolenic acid (LA), gamma-linolenic acid (GLA), and dihomo-gamma-linolenic acid (DGLA) to a patient diagnosed with a cyclooxygenase-2 (Cox-2) enzyme over-expressing disease, wherein said patient is receiving or will receive a primary treatment for the Cox-2 enzyme over-expressing disease;
after expiration of the first period of time, administering for a second period of time a second dosage form comprising gamma-linolenic acid (GLA) to the patient.

2. The method of claim 1, wherein the first period of time is at least two weeks, at least six week, six weeks to three months, or until the patient's red blood cells have an average percent EPA at or above a specified level.

3. (canceled)

4. The method of claim 1, wherein the first period of time precedes initiation of the primary treatment.

5. The method of claim 1, wherein the second period of time is for at least the duration of the primary treatment.

6. The method of claim 1, wherein the second dosage form is not administered to the patient during the first period of time.

7. The method of claim 1, wherein the disease comprises an inflammatory disorder or a cancer.

8. The method of claim 1, further comprising determining or having determined that the patient has a disease that over-expresses the Cox-2 enzyme.

9. The method of claim 1, wherein the primary treatment comprises the patient receiving a cyclooxygenase-2 (COX-2) inhibitor, an histone deacetylase (HDAC) inhibitor, or both.

10. The method of claim 1, wherein the EPA is provided as an ester, triglyceride, free fatty acid, phospholipid or other polar lipid, or combinations thereof or wherein the EPA is provided as a precursor or prodrug of EPA that metabolizes in vivo to form EPA in the patient; wherein the GLA is provided as an ester, triglyceride, free fatty acid phospholipid or other polar lipid, or combinations thereof or wherein the GLA is provided as a precursor or prodrug of GLA that metabolizes in vivo to form GLA in the patient; or both.

11. The method of claim 1, wherein a daily dose of EPA during the first period of time is 100 mg to 500 mg, 500 mg to 2000 mg, or 50 mg to 2000 mg.

12. The method of claim 1, wherein a daily dose of GLA during the second period of time is 100 mg to 500 mg, 500 mg to 2000 mg, 2000 mg to 4000 mg, or 50 mg to 4000 mg.

13. The method of claim 1, wherein a daily dose of EPA during the second period of time is 100 mg to 500 mg, 500 mg to 2000 mg, or 50 mg to 2000 mg.

14. The method of claim 1, wherein the first dosage form, the second dosage form, or both, further comprises docosahexaenoic acid (DHA).

15. The method of claim 1, wherein the first dosage form is administered with the second dosage form during the second period of time.

16. The method of claim 1, wherein the patient is a mammal, a human, a canine, or a feline.

17. A method of adjunctive therapy, the method comprising:
administering for a first period of time a first dosage form comprising eicosapentaenoic acid (EPA) to a patient, wherein the first dosage form substantially excludes linolenic acid (LA), gamma-linolenic acid (GLA), and dihomo-gamma-linolenic acid (DGLA), wherein said patient is receiving or will receive a cyclooxygenase-2 (COX-2) inhibitor, an histone deacetylase (HDAC) inhibitor, or both;
after expiration of the first period of time, administering for a second period of time a second dosage form comprising gamma-linolenic acid (GLA) to the patient.

18. The method of claim 17, wherein the patient has been diagnosed with a cancer or chronic obstructive pulmonary disease (COPD).

19. The method of claim 17, further comprising determining or having determined that the patient has a disease responsive to a cyclooxygenase-2 (COX-2) inhibitor, an HDAC inhibitor, or both.

20. A method of promoting 8-hydroxyoctanoic acid (8-HOA) production, the method comprising:
administering for a first period of time a first dosage form comprising eicosapentaenoic acid (EPA) to a patient and substantially excluding linolenic acid (LA), gamma-linolenic acid (GLA), and dihomo-gamma-linolenic acid (DGLA);
after expiration of the first period of time, administering for a second period of time a second dosage form comprising gamma-linolenic acid (GLA) to the patient.
